(19) United States
(12) Patent Application Publication
(10) Pub. No.: US 2017/0143222 A1
(43) Pub. Date: May 25, 2017

(54) SYSTEM AND METHOD FOR PROVIDING A GRADIENT ATRIAL FIBRILLATION GRAPH

(71) Applicant: Medicomp, Inc., Melbourne, FL (US)

(72) Inventors: Tary Mann, Satellite Beach, FL (US); Anish Sobitharajan Mallika, Thiruvananthapuram (IN); Deepak Hariharan, Trivandrum (IN); Sreehari Krishnankutty, Trivandrum (IN); Arunlal Ramachandran Nair, Trivandrum (IN); Aruna Padmaja Sreekandan, Trivandrum (IN)

(73) Assignee: Medicomp, Inc., Melbourne, FL (US)

(21) Appl. No.: 15/360,166

(22) Filed: Nov. 23, 2016

Related U.S. Application Data
(60) Provisional application No. 62/258,803, filed on Nov. 23, 2015.

(51) Int. Cl. A61B 5/044; A61B 5/04; A61B 5/046

(52) U.S. Cl. CPC A61B 5/044; A61B 5/046; A61B 5/04012

(57) ABSTRACT

A system for displaying biometric measurements may include a first unit of time along a first axis, a second unit of time along a second axis, and a gradient block. The second unit of time may be smaller than the first unit of time. The gradient block may have a primary characteristic indicative of a primary characteristic of a cardiac event detected during a collection time and located at the interaction of the first axis and the second axis corresponding to a collection time of cardiac activity.

### RELATED APPLICATIONS

This application claims the benefit under 35 U.S.C. §119(e) of U.S. Provisional Patent Application Ser. No. 62/258,803 (Attorney Docket Number 612.00074), filed on Nov. 23, 2015, the entire contents of which are incorporated herein by reference.

### FIELD OF THE INVENTION

The present invention relates to the field of medical monitoring and, more specifically, to systems and methods for providing information related to patient cardiac events, particularly, atrial fibrillation, captured during electrocardiogram (ECG) monitoring.

### BACKGROUND

An individual's cardiac cycle consists of a number of stages under healthy physiological and anatomical conditions. Each recognizable component of the bioelectric signals generated by cardiac activity is labeled by convention. The entire cardiac cycle under normal conditions consists of a P-wave, a QRS complex, a T-wave and, in some cases, a U-wave. Each of these components represents a different stage in the cardiac cycle. The P-wave is representative of normal atrial depolarization, or contraction. When functioning properly, the atria of the heart receive blood either form the vena cava or from the pulmonary vein and pump it into their respective ventricles. Under abnormal conditions, specifically atrial fibrillation, the otherwise characteristic P-wave is no longer observable via monitoring equipment. Instead, an almost sinusoidal waveform is present between the T and Q waves. This abnormal waveform is indicative of a state of inefficiency of the atrial pumping mechanism, which causes abnormal flow characteristics in blood within the atria. The natural biological response of blood constituents to such Irregular flow characteristics is to innate the clotting factor cascade, which causes clots to form within the heart. Due to this fact atrial fibrillation is conducive to producing blood clots that could lead to stroke, embolism, and other serious medical conditions; therefore, detection and total evaluation of this condition is extremely important.

A patient's cardiac cycle can be observed with the use of any standard electrocardiography (ECG) equipment. Traditionally, a patient would be required to go to his or her physician's office in order to undergo such testing. However, many advances in the field have allowed for these monitoring systems to be made ambulatory.

Conventional systems used to monitor atrial fibrillation burden on a patient involve the implementation of a single triggering threshold. These currently used systems will only record atrial fibrillation burden wherein a given atrial fibrillation event lasts for a minimum of a certain specified duration.

These monitoring systems and the data they collect and analyze are used by physicians to develop and modify treatments for atrial fibrillation and other cardiac conditions, a medical practice commonly known as titration. The timing and specific duration of atrial fibrillation events play a significant role in the development of these treatment procedures.

Current methods of analysis of atrial fibrillation data are limited by the implemented systems mentioned above. Conventional systems and methods are only able to provide generalized data limited to overall duration of atrial fibrillation burden events which would be qualified under the specified threshold. This shortcoming leaves the recipients of the data with only a crude understanding of a given patient's condition

This background information is provided to reveal information believed by the applicant to be of possible relevance to the present invention. No admission is necessarily intended, nor should be construed, that any of the preceding information constitutes prior art against the present invention.

### SUMMARY OF THE INVENTION

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that when executed by data processing apparatus, cause the apparatus to perform the actions.

One general aspect includes a system for displaying biometric measurements including: a first unit of time along a first axis. The system also includes a second unit of time, smaller than the first unit of time, along a second axis. The system also includes a gradient block having a primary characteristic indicative of a primary characteristic of a cardiac event detected during a collection time and located at the intersection of the first axis and the second axis corresponding to a collection time of cardiac activity.

Other embodiments of this aspect include corresponding computer systems, apparatus, and computer programs recorded on one or more computer storage devices, each configured to perform the actions of the methods.

Implementations may include one or mom of the following features. The second axis may have a dynamically adjustable scale. The gradient block may have a secondary characteristic indicative of a secondary characteristic of the cardiac event. The system may further include a plurality of gradient blocks each having a primary characteristic and located at respective intersections of the first axis and second axis corresponding to respective collection times of cardiac activity. Each of the plurality of gradient blocks may be present only if a characteristic of the cardiac activity is above a threshold level. The collection time may have a duration of 3D minutes. The primary characteristic of the gradient block may be shade. A darker shade may correspond to a primary characteristic of the cardiac event of greater medical concern than a lighter shade. A primary characteristic of the cardiac event may be frequency. The cardiac event may include atrial fibrillation. The first unit of lime may include a calendar day. The first axis may include the major axis. The second unit of time may include the time of day. The second axis may include the minor axis. Implementations of the described techniques may include wearable heart monitoring device hardware, a method or process, or computer software on a computer-accessible medium.

### BRIEF DESCRIPTION OF THE DRAWINGS

FIG. 1 is a graphical representation of measured cardiac information in accordance with an embodiment of the present invention.

FIG. 2 is a zoomed out graphical representation of the graphical representation depicted in FIG. 1.

FIG. 3 is a zoomed in graphical representation of the graphical representation depicted in FIG. 1.

FIG. 4 is a flowchart describing a method according to the present invention to create the graphical representation of FIG. 1.

FIG. 5 is a flowchart describing a method to perform step 36 of FIG. 4 in accordance with an embodiment of the present invention.

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the claimed invention.

In this detailed description of the present invention, a person skilled in the art should note that directional terms, such as “above,” “below,” “upper” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a novel means of presenting the frequency, intensity, and type of cardiac events as they occur over a period of lime. This method may be particularly beneficial in presenting information related to anomalous cardiac events. By way of example, and not as s limitation, anomalous cardiac events may include atrial fibrillation, tachycardia, atrial flutter, bradycardia, ventricular fibrillation, or the like.

A patient may wear a heart monitoring device. The heart monitoring device may have one or more sensors for measuring a patient's heart activity The heart, monitoring device may record information associated with electrical signals from the heart. The heart monitoring device may store the measured cardiac information locally or provide it to an external device for storage. The cardiac information may be analyzed and classified. The classification may be made based on one or more characteristics present in the cardiac information. Classifications of the information may correspond to one or more cardiac events, which may be anomalous.

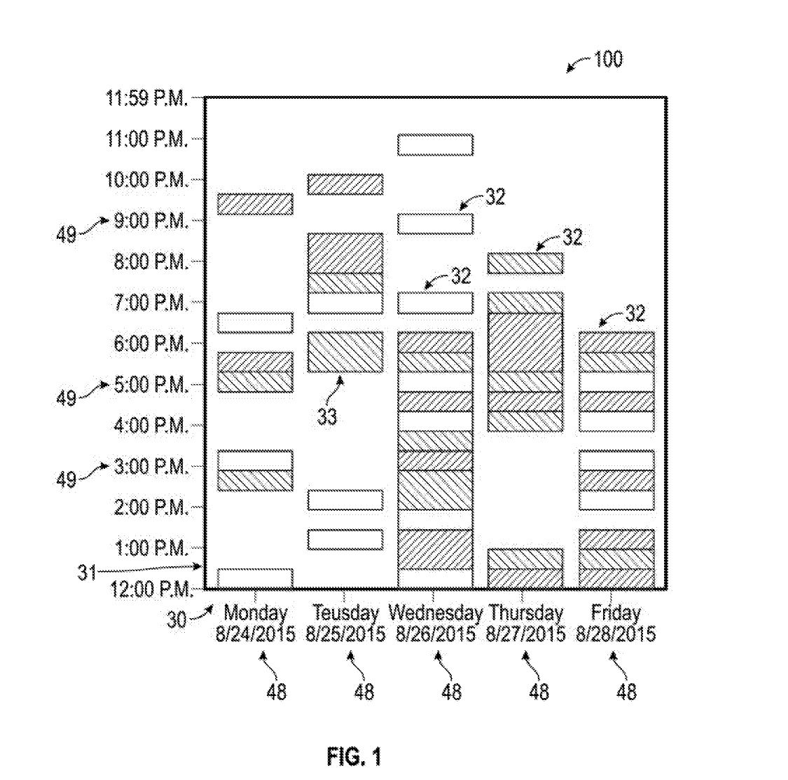

This system 100 may involve the creation or implementation of a graphical representation, which, by way of example, and not as a limitation, may be a table, graph, or the like, to graphically depict the results of the analysis of the cardiac information, as depicted in FIG. 1. The data that populates the graph may be sensed by a physiological signal monitoring system as disclosed in PCT Publication No. WO 2016/168315, which is hereby incorporated by reference in its entirety.

In one embodiment, the major axis 30 may be a first axis and may indicate the calendar day 48, which may be a first unit of time. The minor axis 31 may be a second axis and may indicate the time of day 49, which may be a second unit of time. Each gradient block 32 at the intersection points of the minor axis 31 and the major axis 30 may present information regarding one or more aspect of cardiac activity occurring at that time. In one embodiment, the gradient block 32 may present information related to the percentage or absolute number of cardiac cycles that were classified as anomalous during the time period represented by the gradient block 32. The gradient block 32 may be shaded with differing intensity to correspond with the level of detected anomalous cardiac activity. By way of example, and not as a limitation, a relatively darker shaded gradient block 32 may be located at intersections during which more severe or more frequent anomalous cardiac events occur, if no anomalous activity occurs during a time period, no gradient block 32 may be presented at the corresponding lima period on the graph.

A plurality of shaded gradient blocks 32 may be selected to represent the frequency, seventy, or type of cardiac events. Color, gradient level, fill pattern, or the like of the gradient block 32 may relate to cardiac information recorded at the time corresponding to the location of the gradient block 32. In one embodiment, gradient level of the gradient block 32 may relate to frequency, severity, or type of the associated cardiac event. In another embodiment, color may relate to severity, frequency, or type of cardiac event. In yet another embodiment, fill pattern may relate to severity, frequency, or type of cardiac event. A primary characteristic of the gradient block 32, which may be color, gradient level, fill pattern, or the like, may relate to a primary characteristic of the cardiac event, which may be frequency, severity, or type. A secondary characteristic of the gradient block 32, which may be color, gradient level, fill pattern, or the like, and may be different from the primary characteristic of the gradient block 32, may relate to a secondary characteristic of the cardiac event which may be frequency, severity, or type and may be different from the primary characteristic of the cardiac event. A tertiary characteristic of the gradient block 32, which may be color, gradient level, fill pattern, or the like, and may be different from the primary and secondary characteristics of the gradient block 32, may relate to a tertiary characteristic of the cardiac event, which may be frequency, severity, or type and may be different from the primary and secondary characteristics of the card sac event.

By way of example, and not as a limitation, darker gradient blocks 32 may represent more frequent or more severe cardiac events. In one embodiment, only frequency of a single type of cardiac event may be depicted on the graphical representation. In such an embodiment, no gradient block 32 may be displayed if the cardiac event is not detected during the corresponding time period. There may be gradient blocks 32 of increasing darkness presented to correspond with defection of cardiac events within a range. By way of example, and not as a limitation, if 1-25 cardiac events are detected, the corresponding gradient block 32 may be lighter, if 26-50 cardiac events are detected, the corresponding gradient block 32 may be light, but darker than the lighter gradient block 32, if 51-75 cardiac events are detected, the corresponding gradient block 32 may be dark, and if more than 75 cardiac events are detected, the corresponding gradient block 35 may be darker. In another embodiment only the seventy of a single type of cardiac event may be depicted on the graphical representation. In yet another embodiment, either frequency or severity of a single type of cardiac event may be depicted and represented as a gradient level of the gradient block 32 while the other factor (frequency or severity) may be depicted and represented as a color or fill pattern of the gradient block 32. Those skilled in the art will appreciate that these characteristics are provided for exemplary purposes and other combinations of visual depictions are contemplated and contained within this disclosure.

A plurality of gradient blocks 32 may be disposed on the graphical representation. Each gradient block 32 may depict information obtained from the patient at the time corresponding to the intersection of the major axis 30 with the minor axis 31 at the location at which the gradient block 32 is located. Each gradient block 32 may represent information related to cardiac information obtained from a patient over an interval of time. By way of example, and not as a limitation, each gradient block 32 may represent one minute, two minutes, five minutes, 20 minutes, 30 minutes, one hour, or the like.

By way of example, and not as a limitation, each gradient block may represent a 30 minute period and be located at the corresponding intersection of the minor axis 31 and major axis 30 during which the cardiac information was measured. In embodiments in which the graphical representation is used only to report the occurrence of atrial fibrillation, the number of atrial fibrillation events detected between 6:00 a.m. and 6:30 a.m. on the second day 48 of monitoring may be calculated and displayed at location 33 on the graphical representation, in such an embodiment, the greater the number of atrial fibrillation events recorded during that lime, the darker the gradient block 32 displayed at location 33 would be. Correspondingly, the fewer the number of atrial fibrillation events recorded during that time, the lighter the gradient block 32 displayed at location 33 would be. Similarly, the percentage of detected heartbeats that are classified as atrial fibrillation may be calculated and displayed. If less than a lower threshold number, or threshold seventy, of cardiac events are detected, no gradient block 32 may be present on the graphical representation at the corresponding intersection of the major axis 30 and minor axis 31.

By way of example, and not as a limitation, type of cardiac event frequency of cardiac event, and severity of cardiac event may all be graphically represented using gradient blocks 32. In such an embodiment, the fill pattern of each gradient block 32 may correspond to a different type of cardiac event. The color of each gradient block 32 may correspond to the severity of cardiac events. The gradient level of each gradient block 32 may correspond to the frequency of cardiac events. A legend, or key, may be included with each graphical representation to indicate the cardiac feature related to each visual element of the graphical representation. Those skilled in the art will appreciate that different combinations of visual features and cardiac characteristics are contemplated.

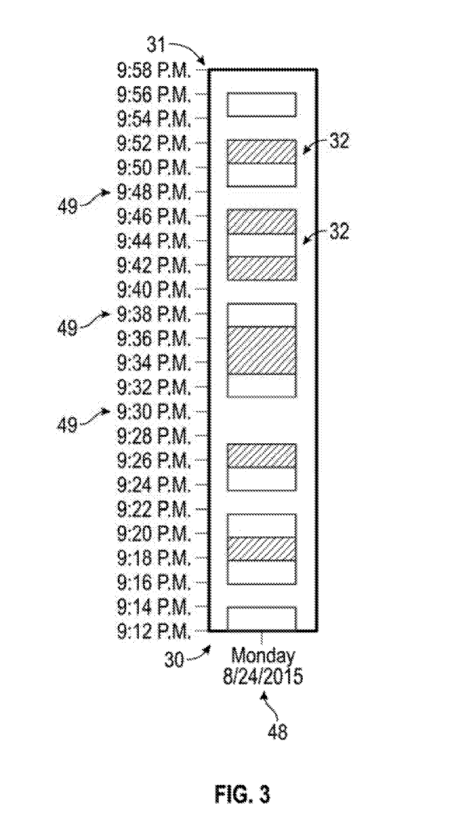

In one embodiment, a viewer of the graphical representation may adjust the timescale being viewed. FIG. 1 depicts Information related to cardiac activity measured from a second unit of time 49 12:00 p.m. to 11:59 p.m. during a first unit of time 48 Monday, Aug. 24, 2015 through Friday, Aug. 28, 2015. FIG. 3 depicts a zoomed in graphical representation of FIG. 1. Specifically, FIG. 3 depicts information related to cardiac activity measured from a second unit of time 49 9:12-10:00 p.m. during a first unit, of time 48 Tuesday, Aug. 25, 2015. In such an embodiment, zooming into the graphical representation may result in viewing a shorter duration of time. Accordingly, each gradient block 32 may represent a smaller duration of time and, therefore, a smaller absolute number of cardiac events. The ability to zoom into and out of the timescale may be referred to as having a dynamically adjustable scale.

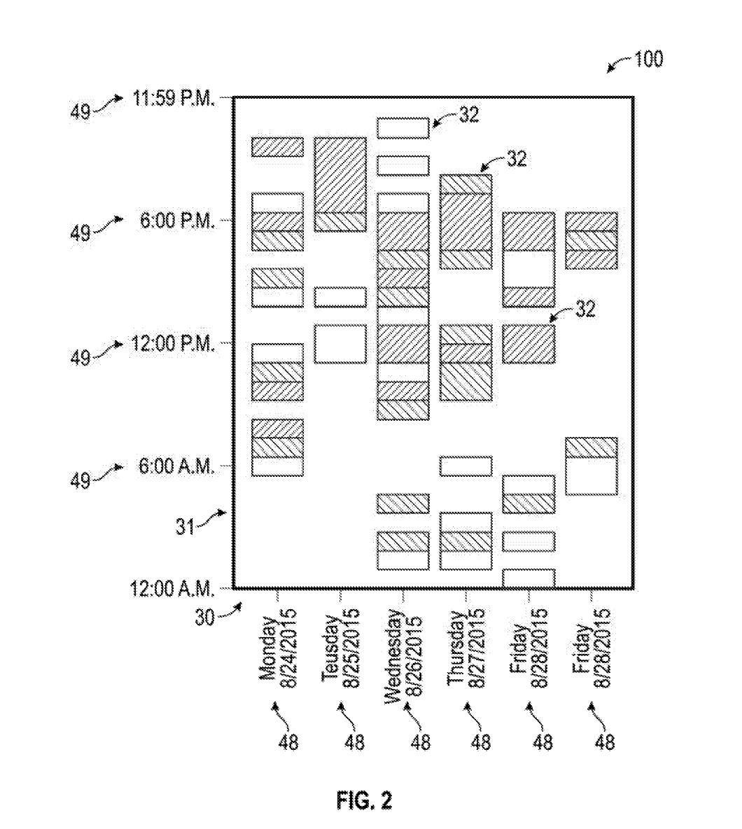

FIG. 2 depicts a zoomed out graphical representation of FIG. 1. Specifically, FIG. 2 depicts information related to cardiac activity measured from a second unit of time 49 12:00 a.m. to 11:59 p.m. during a first unit of time 48 Monday, Aug. 24, 2015 through Sunday, Aug. 30, 2015. Zooming out of the graphical representation may result in viewing a longer duration of time. Accordingly, each gradient block 32 may represent a larger duration of time and, therefore, a larger absolute number of cardiac events.

When changing the timescale being viewed, the gradient level, fill pattern, or color of the gradient block 32 may be altered to reflect the cardiac activity occurring during the time period associated with the gradient block 32.

The graphical representation may Include information related to the total percentage of time the patient experiencing anomalous cardiac events. The graphical representation may be configured to depict the percentage of time the patient spent In a specified anomalous cardiac event type during a specified duration, during the entire monitoring duration, during a configurable range of time, or the like. The percentage of time a patient spends in a cardiac event may be displayed on the graphical representation displaying the time period for which the percentage is calculated.

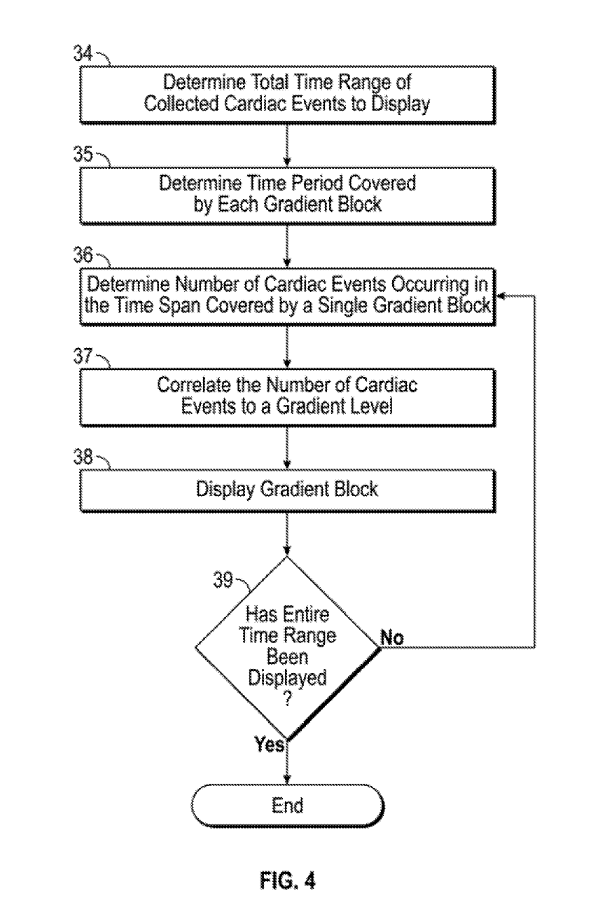

FIG. 4 depicts an embodiment of the Inventive method for creating a graphical representation depicting the frequency of cardiac events. The desired time range of collected cardiac events to be displayed must be determined 34. This may be determined by a system default value, a user selected value, or the like. The time period covered by each gradient block must be determined 35. This may be calculated based on the total time range selected in step 34. The time period covered by each gradient block may be determined by a system default value, a user selected value, or the like. The default time period covered by each gradient block may correspond to the total time range to be displayed. The number of target cardiac events occurring in the time span covered by each gradient block must be determined 38. Starting from the beginning of the total time range to be displayed, through the end of the time period covered by a single gradient block, the total number of relevant cardiac events must be counted. The number of detected cardiac events may be correlated to a gradient level. A gradient block with the corresponding gradient level may be displayed 38. The method must determine whether or not the entire time range has been displayed in gradient blocks 39. If the entire time range has been displayed, the method is complete. If there is a portion of the time range left to be displayed, the number of cardiac events occurring in the time span covered by the next single gradient block may be determined 36.

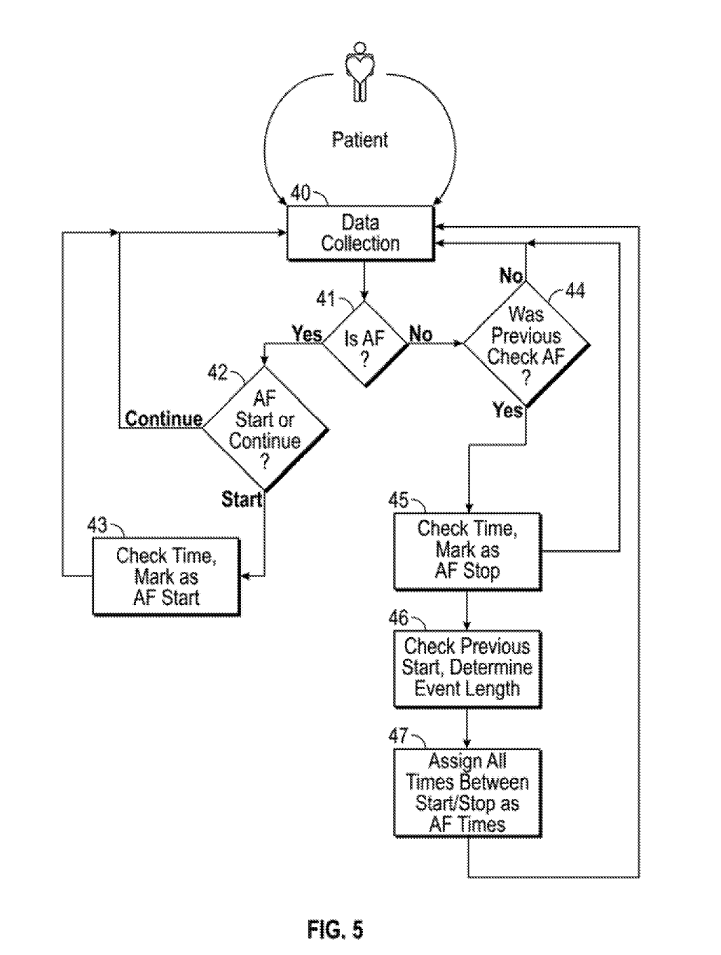

FIG. 5 depicts an embodiment of the inventive method that may be utilized to perform at least a portion of step 36 of FIG. 4, Data is collected from a patient 40. This data may be collected using a biophysical sensor. The sensor may be an electrode. The electrode may measure electrical heart activity. The collected information is analyzed to determine if the patient is experiencing atrial fibrillation 41. If the patient is experiencing atrial fibrillation, the system 100 determines whether or not this is the beginning occurrence of atrial fibrillation or if the previously measured patient waveform was atrial fibrillation 42, In instances in which the measured atrial fibrillation is the beginning of an occurrence of atrial fibrillation, the time is marked as an atrial fibrillation start time 43. In instances in which the measured atrial fibrillation; is a continuing occurrence of atrial fibrillation, the system 100 continues to monitor the patient When atrial fibrillation is no longer defected by the system 100 the system 100 determines whether the previously detected waveform was atrial fibrillation 44. In instances in which the absence of atrial fibrillation occurs after an instance of atrial fibrillation, the time is marked as an atrial fibrillation stop time 45. The length of the atrial fibrillation event is determined utilizing the atrial fibrillation start and end times 48. All time between the start and end times is classified as atrial fibrillation time 47 and the system 100 continues to collect patient data 40, In instances in which the absence of atrial fibrillation occurs immediately alter a previous absence of atrial fibrillation, the system 100 continues to collect patient data 40.

Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference lo exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the scope of the appended claims. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first second, etc. are used to distinguish one element from another. Furthermore, the use of the terms a, an, etc. do not denote a limitation of quantity, but rather denote the presence of at least one of the referenced item.

Thus the scope of the invention should be determined by the appended claims and their legal equivalents, and not by the examples given.

That which is claimed is:

1. A system for displaying biometric measurements comprising:
a first unit of time along a first axis;
a second unit of time, smaller than the first unit of time, along a second axis; and
a gradient, block having a primary characteristic indicative of a primary characteristic of a cardiac event detected during a collection time and located at an intersection of the first axis and the second axis corresponding to a collection time of cardiac activity.

2. The system according to claim 1 wherein the second axis has a dynamically adjustable scale.

3. The system according to claim 1 wherein the gradient block has a secondary characteristic indicative of a secondary characteristic of the cardiac event.

4. The system according to claim 1 further comprising:
a plurality of gradient blocks each having a primary characteristic and located at respective intersections of the first axis and second axis corresponding to respective collection times of cardiac activity.

5. The system according to claim 4 wherein each of the plurality of gradient blocks is present only if a characteristic of the cardiac activity is above a threshold level.

6. The system according to claim 1 wherein the collection time has a duration of 30 minutes.

7. The system according to claim 1 wherein the primary characteristic of the gradient block is shade.

8. The system according to claim 7 wherein a darker shade corresponds to a primary characteristic of a cardiac event, of greater medical concern than a lighter shade.

9. The system according to claim 1 wherein a primary characteristic of the cardiac event is frequency.

10. The system according to claim 1 wherein the cardiac event comprises atrial fibrillation.

11. The system according to claim 1 wherein the first unit of time 48 comprises a calendar day;
wherein the first axis comprises the major axis;
wherein the second unit of time comprises the time of day; and
wherein the second axis comprises the minor axis.

12. A system for displaying biometric measurements comprising:
a first unit of time along a first axis;
a second unit of time, smaller than the first unit of time, along a second axis; and
a plurality of gradient blocks each having a primary characteristic indicative of a primary characteristic of a cardiac event detected during a collection time and located at respective intersections of the first axis and second axis corresponding to respective collection times of cardiac activity; and
wherein each of the plurality of gradient blocks is present only if a characteristic of the cardiac activity is above a threshold level.

13. The system according to claim 12 wherein the second axis has a dynamically adjustable scale.

14. The system according to claim 12 wherein the plurality of gradient blocks has a secondary characteristic indicative of a secondary characteristic of the cardiac event.

15. The system according to claim 12 wherein the collection time has a duration of 30 minutes.

16. The system according to claim 12 wherein the primary characteristic of the plurality of gradient blocks is shade; and
wherein a primary characteristic of the cardiac event is frequency.

17. The system according to claim 16 wherein a darker shade corresponds to a greater frequency than a lighter shade.

18 The system according to claim 12 wherein the cardiac event comprises atrial fibrillation.

19. The system according to claim 12 wherein the first unit of time comprises a calendar day;
wherein the first axis comprises the major axis:
wherein the second unit of time comprises the time of day; and
wherein the second axis comprises the minor axis.

20. A system for displaying biometric measurements comprising;
a first unit of time along a first axis;
a second unit of time, smaller than the first unit of time, along a second axis having a dynamically adjustable scale; and
a plurality of gradient blocks each having a shade indicative of a frequency of detection of atrial fibrillation during a collection time, having a secondary characteristic, indicative of a secondary characteristic of the atrial fibrillation, and located at respective intersections of the first axis and second axis corresponding to respective collection times of cardiac activity;
wherein each of the plurality of gradient blocks is present only if the frequency of the atrial fibrillation detection is above a threshold level; and
wherein a darker shade corresponds to a greater frequency of detection than a lighter shade.
